(19) United States
(12) Patent Application Publication
(10) Pub. No.: US 2008/0125331 A1
(43) Pub. Date: May 29, 2008

(54) MAGENTA LOW FLUORESCENCE DYE FOR COATED OPTICAL BEAD RANDOM ARRAY DNA ANALYSIS

(76) Inventors: Krishnan Chari, Fairport, NY (US); Tiecheng A. Qiao, Webster, NY (US); Donald R. Diehl, Rochester, NY (US); Samuel Chen, Penfield, NY (US); Kevin W. Williams, Rochester, NY (US); David A. Stegman, Churchville, NY (US)

Correspondence Address: Susan L. Parulski;Patent Legal Staff, Carestream Health, Inc., 150 Verona Street, Rochester, NY, 14608, US

(21) Appl. No.: 11/945,484

(22) Filed: Nov. 27, 2007

Related U.S. Application Data
(62) Division of application No. 10/713,522, filed on Nov. 14, 2003.

(51) Int. Cl. C40B 40/16; C12Q 1/68

(52) U.S. Cl. 506/21; 435/6

(57) ABSTRACT

A coating composition for making a protein microarray, the composition comprising a gelling agent or a precursor to a gelling agent, and microspheres; the microspheres containing a dye represented by the Formula (1):

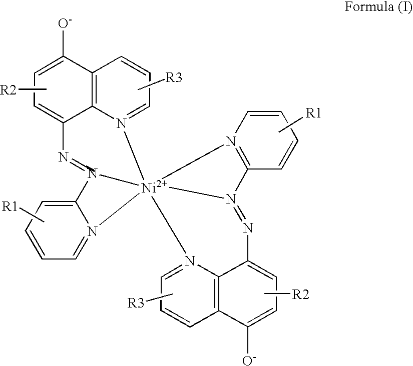

wherein: R1=one or more substitutent selected from the group of H, chloro, alkoxycarbonyl, arylsulfamoyl, or alkylsulfamoyl; R2=one or more substituent selected from the group of H, carboxamido, or alkoxycarbonyl; R3=one or more substituent selected from the group of H, chloro, substituted or unsubstituted alkyl, aryl, carboxamido, or alkoxycarbonyl.

### CROSS-REFERENCE TO RELATED APPLICATIONS

This is a Divisional of commonly-assigned copending U.S. patent application Ser. No. 10/713,522, entitled MAGENTA LOW FLUORESCENCE DYE FOR COATED OPTICAL BEAD RANDOM ARRAY DNA ANALYSIS, filed on Nov. 14, 2003.

Reference is made to commonly-assigned, copending U.S. patent application Ser. No. 10/713,165, entitled CYAN LOW FLUORESCENCE DYE FOR COATED OPTICAL BEAD RANDOM ARRAY DNA ANALYSIS and copending U.S. patent application Ser. No. 10/713,246, entitled YELLOW LOW FLUORESCENCE DYE FOR COATED OPTICAL BEAD RANDOM ARRAY DNA ANALYSIS, both filed on Nov. 14, 2003. Reference is also made to U.S. Publication No. 2003/0068609 (U.S. patent application Ser. No. 09/942,241), filed Aug. 29, 2001, entitled RANDOM ARRAY OF MICROSPHERES. The copending applications are incorporated by reference herein for all that they contain.

### FIELD OF THE INVENTION

The present invention concerns biological microarray technology in general. In particular, the invention concerns the coloration of polystyrene microspheres or “beads” coated on a substrate to form a microarray.

### BACKGROUND OF THE INVENTION

Ever since they were invented in the early 1990s (Science, 251, 767-773, 1991), high-density arrays formed by spatially addressable synthesis of bioactive probes on a 2-dimensional solid support have greatly enhanced and simplified the process of biological research and development. The key to current microarray technology is deposition of a bioactive agent at a single spot on a microchip in a “spatially addressable” manner.

Current technologies have used various approaches to fabricate microarrays. For example, U.S. Pat. Nos. 5,412,087, and 5,489,678 demonstrate the use of a photolithographic process for making peptide and DNA microarrays. The patent teaches the use of photolabile protecting groups to prepare peptide and DNA microarrays through successive cycles of deprotecting a defined spot on a 1 cm×1 cm chip by photolithography, then flooding the entire surface with an activated amino acid or DNA base. Repetition of this process allows construction of a peptide or DNA microarray with thousands of arbitrarily different peptides or oligonucleotide sequences at different spots on the array. This method is expensive. An ink jet approach is being used by others (e.g., U.S. Pat. Nos. 6,079,283; 6,083,762; and 6,094,966) to fabricate spatially addressable arrays, but this technique also suffers from high manufacturing cost in addition to the relatively large spot size of 40 to 100 μm.

Because the number of bioactive probes to be placed on a single chip usually runs anywhere from 1,000 to 100,000 probes, the spatial addressing method is intrinsically expensive regardless how the chip is manufactured. An alternative approach to the spatially addressable method is the concept of using fluorescent dye-incorporated polymeric beads to produce biological multiplexed arrays. U.S. Pat. No. 5,981,180 discloses a method of using color coded beads in conjunction with flow cytometry to perform multiplexed biological assay. Microspheres conjugated with DNA or monoclonal antibody probes on their surfaces were dyed internally with various ratios of two distinct fluorescence dyes. Hundreds of “spectrally addressed” microspheres were allowed to react with a biological sample and the “liquid array” was analyzed by passing a single microsphere through a flow cytometry cell to decode sample information. U.S. Pat. No. 6,023,540 discloses the use of fiber-optic bundles with pre-etched microwells at distal ends to assemble dye loaded microspheres. The surface of each spectrally addressed microsphere was attached with a unique bioactive agent and thousands of microspheres carrying different bioactive probes combined to form “beads array” on pre-etched microwells of fiber optical bundles. More recently, a novel optically encoded microsphere approach was accomplished by using different sized zinc sulfide-capped cadmium selenide nanocrystals incorporated into microspheres (Nature Biotech. 19, 631-635, (2001)). Given the narrow band width demonstrated by these nanocrystals, this approach significantly expands the spectral barcoding capacity in microspheres. Further, it is reported in WO 02/077291 (Oct. 3, 2002) that beads used in the manufacture of microarrays may be colored with materials described as fluorophores or chromophores, in which WO 02/077291 defines fluorophore as a material which absorbs light and later emits light of a different wavelength, and chromophores as those materials which absorb light and do not emit light but instead convert the light into heat. It is reported in WO 02/077291 that the dyes imbibed in microspheres may be of the chromophore type but are preferred to be of the fluorophore type for the application described in WO 02/077291. Indeed, WO 02/077291 reports only the use of fluorophores in the examples described within the International Publication.

Even though the “spectrally addressed microsphere” approach does provide an advantage in terms of its simplicity over the old fashioned “spatially addressable” approach in microarray making, there are still needs in the art to make the manufacture of biological microarrays less difficult, less expensive, and to provide better dyes for coloration of microspheres.

### SUMMARY OF THE INVENTION

The present invention provides a dye for coloring microspheres magenta, i.e.—green light absorbing, with colorant materials that have the property of very low fluorescence intensity such that the resultant colored microspheres do not substantially fluoresce when excited by visible light.

The present invention also provides a coating composition for making a protein microarray, the composition comprising a gelling agent or a precursor to a gelling agent and microspheres; the microspheres containing a dye represented by Formula (I):

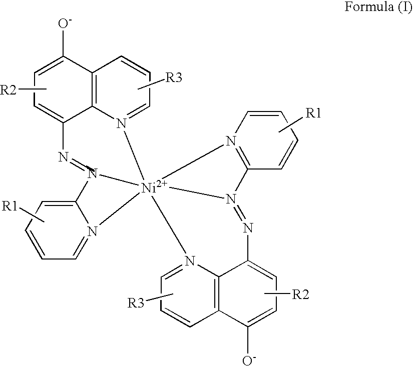

The invention utilizes a unique coating composition and technology to prepare a microarray on a substrate that may or may not be pre-etched with microwells and need not be premarked in any way with sites to attract the colored microspheres, as disclosed in the art. By providing the option of using unmarked substrates or substrates that need no pre-coating preparation, the present invention provides a huge manufacturing advantage compared to the existing technologies that do not provide the option. The invention discloses a method whereby color addressable mixed beads in a unique composition can be randomly distributed on a substrate that has no wells or sites to attract the microspheres.

The present invention provides a microarray that is less costly and easier to prepare than those previously disclosed, and further can be used in a colored microarray device such as described herein wherein green light absorbance is desired to be maximized and fluorescence of the dye imbibed in the colored polystyrene microsphere bead is desired to be minimized.

### BRIEF DESCRIPTION OF THE DRAWINGS

FIG. 1 is a diagram of a coated microsphere array device containing yellow colored microspheres 1, magenta colored microspheres 2 and cyan colored microspheres 3.

FIG. 2 is a schematic of a microspectrometer and fluorescence detection system used to characterize the colorant dye loaded in the microspheres.

FIG. 3 is a graph showing a spectroscopic response of MD-1 Dye loaded in a microsphere.

### DETAILED DESCRIPTION OF THE INVENTION

The coloration of a polystyrene microsphere bead requires that colorant materials are soluble in an organic solvent mixture that is designed to swell, but not dissolve, the polystyrene microsphere bead. Further, the colorant material must migrate from the solvent mixture into the polystyrene bead rendering the bead with high coloration. Further, the colorant must remain in the microsphere bead during the process of bead filtration, solvent removal and subsequent washing and de-swelling of the polystyrene beads. Organic colorant materials with high solubility in a mixture of acetone and toluene have been found to be most suitable to meet these requirements. In order for the dyes to impart strong coloration to the polystyrene microsphere beads it is also desirable that the colorant materials possess a high extinction coefficient. For the application described herein it is further desirable that the colorant material possess the property of no detectable fluorescence upon light exposure when imbibed in a polystyrene matrix. It is further desirable that the colorant materials possess such properties which impart a non-fading color to the polystyrene beads, and that the dye materials be easy to synthesize, and of low cost.

Those skilled in the art of colorant technology will recognize the difficulty of meeting all the requirements stated above. There are many classes of dyes and pigments known to the colorant art. Solubility in water, or organic solvents, has been a topic of high interest in the past for a wide variety of colorant applications, thus mathematical parameters have been devised to help the colorant scientist understand and predict dye or pigment solubility. However, there are no general guideline parameters with which a colorant scientist may predict the fluorescence of any given colorant material. Therefore, the colorant scientist must undertake an empirical approach to the discovery of colorant materials that are non-fluorescent. It appears that dye materials containing a specific halogen functionality are particularly likely to possess the property of very low fluorescence. Thus, the dyes of this invention have been found to have good solubility in the organic solvents required for bead coloration, high extinction, and remarkably low fluorescence when imbibed in a polystyrene microsphere bead.

The dyes of this invention that meet the above requirements for coloring polystyrene microsphere magenta, and have been found to possess the property of very low fluorescence, are described by the general formula I below and more preferably by the general formula II below:

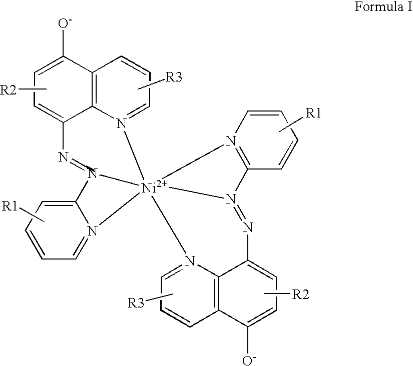

wherein: R1=one or more substitutent selected from the group of H, chloro, alkoxycarbonyl, arylsulfamoyl, or alkylsulfamoyl;

R2=one or more substituent selected from the group of H, carboxamido, or alkoxycarbonyl;

R3=one or more substituent selected from the group of H, chloro, substituted or unsubstituted alkyl, aryl, carboxamido, or alkoxycarbonyl.

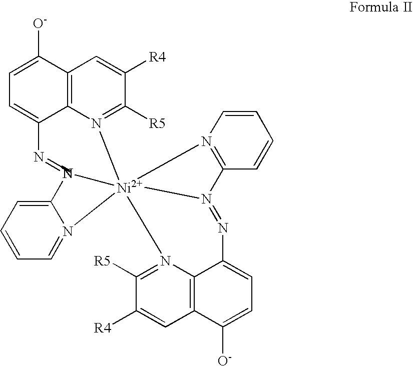

wherein: R4=alkoxycarbonyl; and

R5=H, chloro, or alkyl.

Dyes of the type described above are known to those skilled in the art of colorant science. These dyes, known as nickel metallized pyridylazoquinolinol dyes, have been reportedly useful in the areas of photographic science and ink jet printing. We have found that these dyes surprisingly possess the quality of good solubility in solvents suitable for coloring polystyrene beads, and further specifically those dyes which possess alkoxycarbonyl substitution on the quinolinol portion of the dye chromophore have the very desirable property of extremely low fluorescence when imbibed into the polystyrene microsphere beads.

Examples of dyes that fulfill the requirements set out above are presented below, but one skilled in the art will recognize that the present invention is not limited thereto. Synthesis of dyes of this invention may be achieved by methods known in the dye art as described in, for example: US2002086112, EP 1127707 and U.S. Pat. No. 4,420,550, and by the method described in the Experimental Section herein.

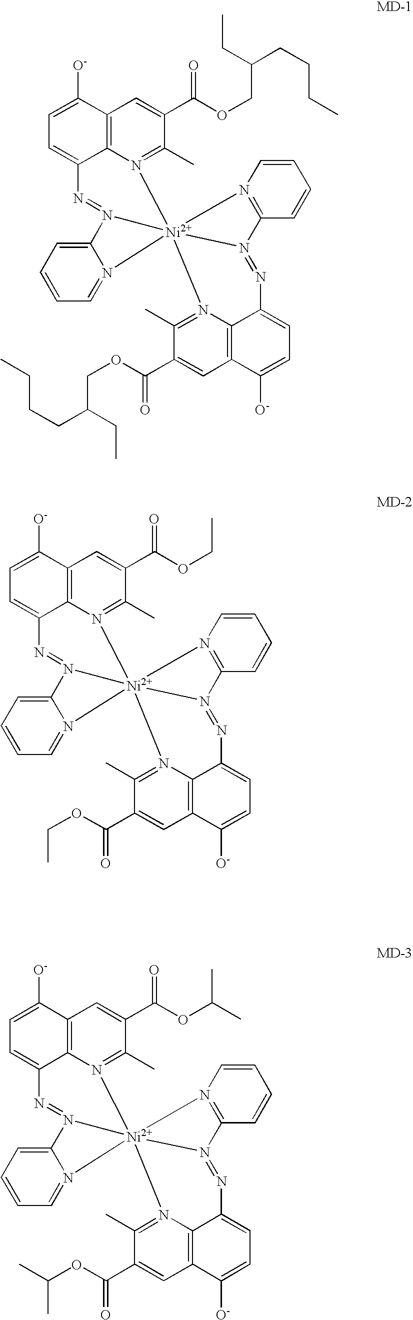

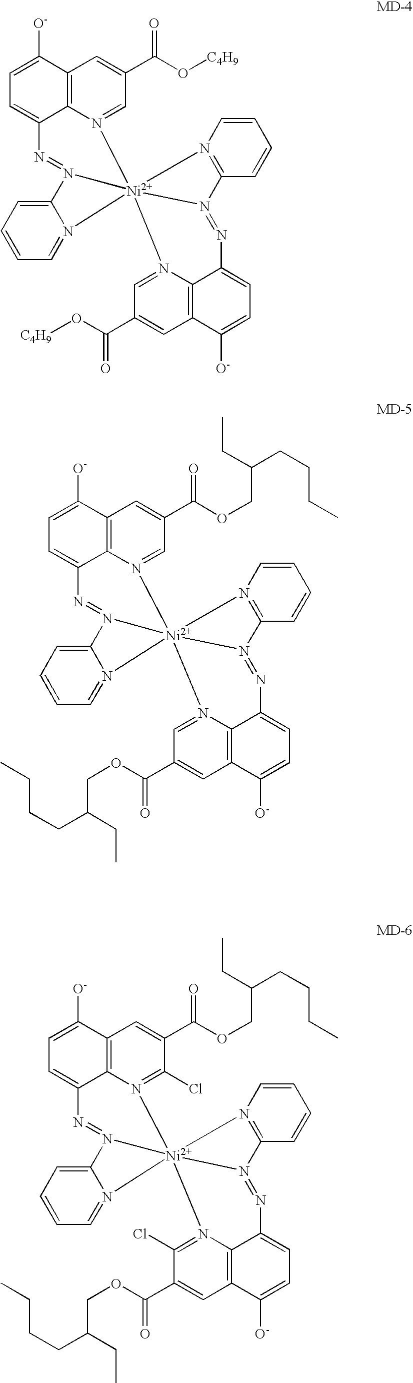

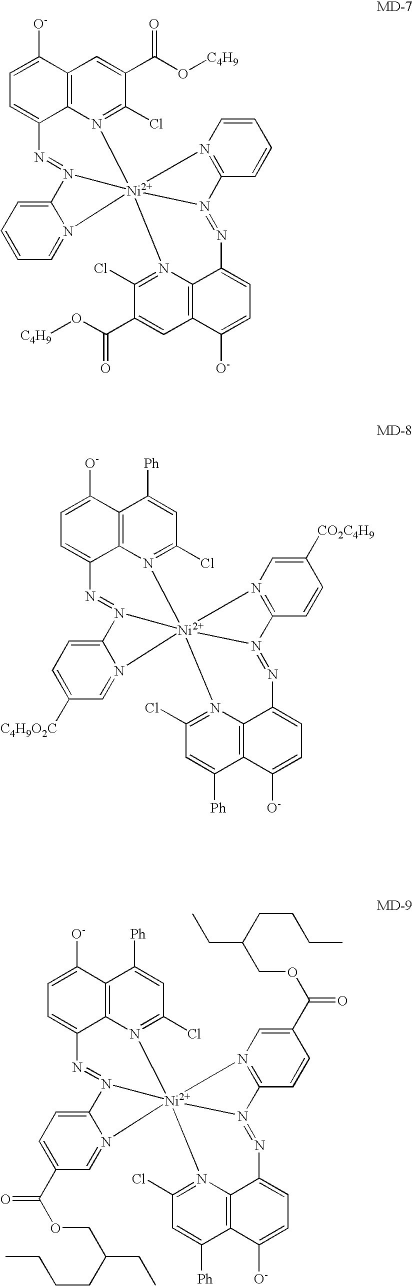

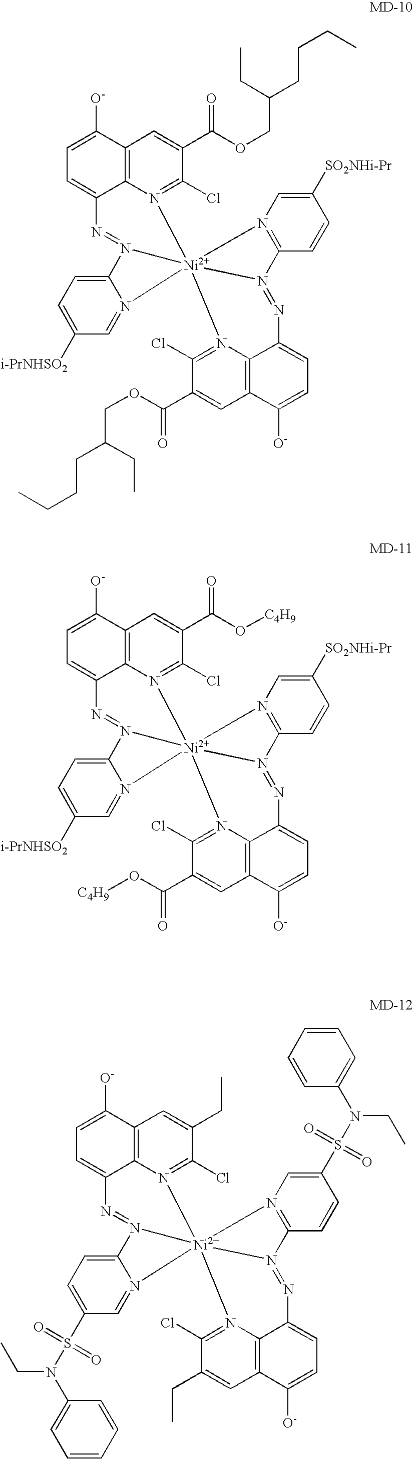

As used herein, the term “sol-to-gel transition” or “gelation” means a process by which fluid solutions or suspensions of particles form continuous three-dimensional networks that exhibit no steady state flow. This can occur in polymers by polymerization in the presence of polyfunctional monomers, by covalent cross-linking of a dissolved polymer that possesses reactive side chains and by secondary bonding, for example, hydrogen bonding, between polymer molecules in solution. Polymers such as gelatin exhibit thermal gelation that is of the latter type. The process of gelation or setting is characterized by a discontinuous rise in viscosity. (See, P. I. Rose, “The Theory of the Photographic Process”, 4^{th} Edition, T. H. James ed. pages 51 to 67).

As used herein, the term “gelling agent” means a substance that can undergo gelation as described above. Examples include materials such as gelatin, water-soluble cellulose ethers or poly(n-isopropylacrylamide) that undergo thermal gelation or substances such as poly(vinyl alcohol) that may be chemically cross-linked by a borate compound. Other gelling agents may be polymers that may be cross-linked by radiation such as ultraviolet radiation. Examples of gelling agents include acacia, alginic acid, bentonite, carbomer, carboxymethylcellulose sodium, cetostearyl alcohol, colloidal silicon dioxide, ethylcellulose, gelatin, guar gum, hydroxyethylcellulose, hydroxypropyl cellulose, hydroxypropyl methylcellulose, magnesium aluminum silicate, maltodextrin, methylcellulose, polyvinyl alcohol, povidone, propylene glycol alginate, sodium alginate, sodium starch glycolate, starch, tragacanth and xanthum gum. (For further discussion on gelling agents, see, accompanying reference Secundum Artem, Vol. 4, No. 5, Lloyd V. Allen). A preferred gelling agent is alkali pretreated gelatin.

As used herein, the term “random distribution” means a spatial distribution of elements showing no preference or bias. Randomness can be measured in terms of compliance with that which is expected by a Poisson distribution.

The present invention teaches a composition and a method for making a random array of colored microspheres, also referred to as “colored beads”, on a substrate. The distribution or pattern of the colored microspheres on the substrate is entirely random and the colored microspheres are not attracted or held to sites that are pre-marked or predetermined on the substrate as in other methods previously disclosed. In the present invention, the colored microspheres are immobilized randomly when the gelling agent in which they are carried undergoes a sol-to-gel transition.

The invention discloses a polymeric latex bead based random microarray with each colored bead in the array having a distinct signature that would distinguish the colored bead. Such a signature may be based on color, shape or size of the bead. For signatures based on color, the color may be derived from mixing three dyes representing the visual colors yellow, magenta, and cyan to create thousands of distinguishable beads with distinct “color addresses” (unique RGB values, e.g. R=0, G=204, B=153). The beads can be made with sites on their surface that are “active”, meaning that at such sites physical or chemical interaction can occur between the colored bead and other molecules or compounds. Such compounds may be organic or inorganic. Usually, the molecule or compound is organic—nucleic acid, protein or fragments thereof, are examples. To the surface of each color coded bead may be attached a pre-synthesized oligonucleotide, a monoclonal antibody, or any other biological agents. Therefore, each color address can correspond to a specific bioactive probe. These colored beads may be mixed in equal amounts, and the random microarray fabricated by coating the mixed beads in a single or multilayer format.

Coating methods are broadly described by Edward Cohen and Edgar B. Gutoff in Chapter 1 of “Modern Coating And Drying Technology”, (Interfacial Engineering Series; v. 1), (1992), VCH Publishers Inc., New York, N.Y. For a single layer format, suitable coating methods may include dip coating, rod coating, knife coating, blade coating, air knife coating, gravure coating, forward and reverse roll coating, and slot and extrusion coating.

Fluorescently/chemiluminescently labeled biological sample can be hybridized to the bead based random microarray. The signals from both “color addressable” polymeric beads and biological sample non-selectively labeled with fluorescence/chemiluminescence may be analyzed by a charge coupled device after image enlargement through an optical system. The recorded array image can be automatically analyzed by an image processing algorithm to obtain bioactive probe information based on the RGB color code of each bead, and the information compared to the fluorescence/chemiluminescence image to detect and quantify specific biological analyte materials in the sample. Optical or other electro-magnetic means may be applied to ascertain signature.

Although microspheres or particles having a substantially curvilinear shape are preferred because of ease of preparation, particles of other shape such as ellipsoidal or cubic particles may also be employed. Suitable methods for preparing the particles are emulsion polymerization as described in “Emulsion Polymerization” by I. Piirma, Academic Press, New York (1982) or by limited coalescence as described by T. H. Whitesides and D. S. Ross in J. Colloid Interface Science, vol. 169, pages 48-59, (1985). The particular polymer employed to make the particles or microspheres is a water immiscible synthetic polymer that may be colored. The preferred polymer is any amorphous water immiscible polymer. Examples of polymer types that are useful are polystyrene, poly(methyl methacrylate) or poly(butyl acrylate). Copolymers such as a copolymer of styrene and butyl acrylate may also be used. Polystyrene polymers are conveniently used. The formed microsphere is colored using an insoluble colorant that is a pigment or dye that is not dissolved during coating or subsequent treatment. Suitable dyes may be oil-soluble in nature. It is preferred that the dyes are non-fluorescent when incorporated in the microspheres.

The microspheres are desirably formed to have a mean diameter in the range of 1 to 50 microns; more preferably in the range of 3 to 30 microns and most preferably in the range of 5 to 20 microns. It is preferred that the concentration of microspheres in the coating is in the range of 100 to a million per cm², more preferably 1000 to 200,000 per cm² and most preferably 10,000 to 100,000 per cm².

The attachment of bioactive agents to the surface of chemically functionalized microspheres can be performed according to the published procedures in the art (Bangs Laboratories, Inc, Technote #205). Some commonly used chemical functional groups include, but not limited to, carboxyl, amino, hydroxyl, hydrazide, amide, chloromethyl, epoxy, aldehyde, etc. Examples of bioactive agents include, but are not limited to, oligonucleotides, DNA and DNA fragments, PNAs, peptides, antibodies, enzymes, proteins, and synthetic molecules having biological activities.

### EXAMPLES

### Example 1

### Synthesis of Dye MD-1

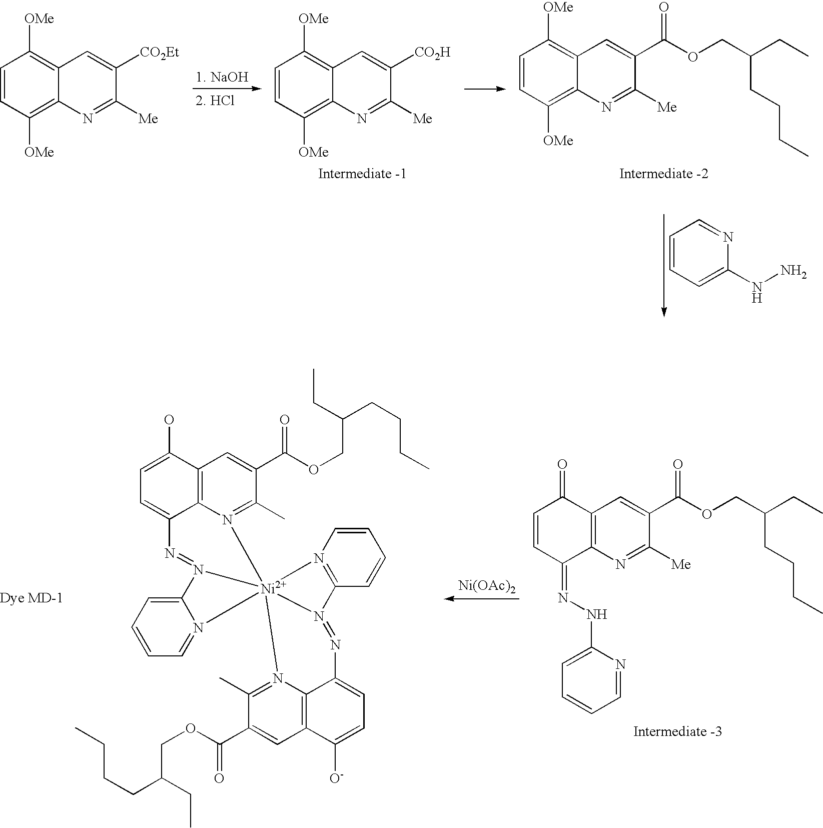

### Synthesis of Intermediate-1

Into a 3 liter round bottom flask was placed 99 grams (0.36 moles) of ethyl 5,9-dimethoxy-2-methyl-3-quinolinecarboxylate (prepared according to the procedure of Heterocycles (1993) 36 (6) 1387-1398) and 1 liter of ethanol. To this stirred mixture was added a solution of 60 grams of 50% sodium hydroxide in 500 mL of water. The stirred reaction was heated to reflux for 2 hours then cooled. The ethanol was removed at reduced pressure and the residue was chilled to 0° C. then 130 mL of concentrated hydrochloric acid was added to achieve a pH of 3. The mixture was allowed to stand at 5° C. overnight then the yellow solid product was collected, washed with cold water, and dried to give 50 grams of Intermediate-1. Structure confirmed by NMR; HPLC purity 99%.

### Synthesis of Intermediate-2.

In a 1 liter round bottom flask was placed 49 grams (0.2 moles) of Intermediate-1 and 500 mL of dichloromethane. To the stirred mixture was added 111 mL (0.8 moles) of triethylamine. After stirring 15 minutes, the solution was chilled to 0° C. and 58 grams (0.3 moles) of 2-ethylhexyl chloroformate was added dropwise while maintaining a temperature of 5° C. The reaction was stirred at 0° C. for 45 min then 39 grams (0.3 moles) of 2-ethyl-1-hexanol was added over 10 minutes. The reaction was stirred at room temperature for 4 hours, then the dichloromethane was removed under reduced pressure and the residue dissolved in 250 mL of methanol. To the slowly stirred solution was added 500 mL of water slowly until a precipitate formed. The solid was collected, washed with water, and dried to give 56 grams of Intermediate-2. Structure confirmed by NMR; HPLC purity 98%.

### Synthesis of Intermediate-3.

In a 3 liter round bottom flask was added 36 grams (0.1 moles) of Intermediate-2 and 500 mL of ethanol. The mixture was stirred and cooled to 0° C. then 110 grams (0.2 moles) of ceric ammonium nitrate dissolved in 250 mL of water was added dropwise maintaining 0° C. After 2 hours, 11 grams (0.1 moles) of 2-hydrazinopyridine dissolved in 208 mL of 5% hydrochloric acid was added dropwise at 0° C. The reaction was stirred and allowed to warm to 20° C. over 2 hours. Then 40 grams of sodium acetate dissolved in 250 mL of water was added. The precipitated product was collected, washed with water, then slurried twice at room temperature in methanol (2 mL/gram), collected and dried under high vacuum to give 20 grams of Intermediate-3. Structure confirmed by NMR; HPLC purity 99.5%.

### Synthesis of Dye MD-1.

Into a 1 liter round bottom flask was placed 13 grams (0.03 moles) of Intermediate-3 and 250 mL of ethanol and stirred to dissolve at room temperature. To this stirred solution was added a filtered solution of 3.8 grams (0.015 moles) of nickel acetate tetrahydrate in 80 mL of water. The mixture was stirred for 48 hours in the dark then 170 mL of water was added. The resulting green solid was collected by filtration then recrystallized by dissolving in 100 mL of ethanol at boiling and chilling at 5° C. for 3 hours. The product crystals were collected by filtration, washed with cold ethanol then dried under vacuum at 35° C. to give 10 grams of MD-1. HPLC purity 99%; Mass Spectrum: ES+897, ES−895; Absorbance maximum 546 nm (MeOH); Extinction maximum=132,000.

### Example 2

This example illustrates the colorant density obtained by using dye MD-1 (whose synthesis is described above) to color polystyrene micro-sphere beads.

A 4.2% aqueous suspension of polystyrene beads prepared by emulsion polymerization and having a mean size of 10 micrometers was obtained from Interfacial Dynamics Corporation, Portland, Oreg.

A suspension of magenta colored beads was prepared by first dissolving 0.0008 grams of MD-1 in 0.02 grams of toluene and 2 grams of acetone. 5.0 grams of the suspension of non-dyed (clear) polystyrene beads was combined with 3 grams of acetone. This mixture was then added rapidly to the solution of MD-1 in acetone and toluene while stirring to prepare a suspension of colored beads. The suspension of colored beads was then filtered using a porous cotton filter, poured into a dialysis bag (12,000 to 14,000 molecular weight cut off) and washed with distilled water for one hour. After washing, the suspension of colored beads was filtered again using a porous cotton filter.

Spectroscopic responses and fluorescence intensity levels in colorant-dyed microspheres were analyzed using a hybrid analytical system comprising three parts: optical microscope, fluorescence microscope, and ultra-violet visible (UV-VIS) micro-spectrometer. This system uses high-intensity light, lenses, mirrors, apertures and optical detectors to not only generate a magnified image of the coated array of microspheres, but also selectively capture the spectroscopic response of individual colored microspheres.

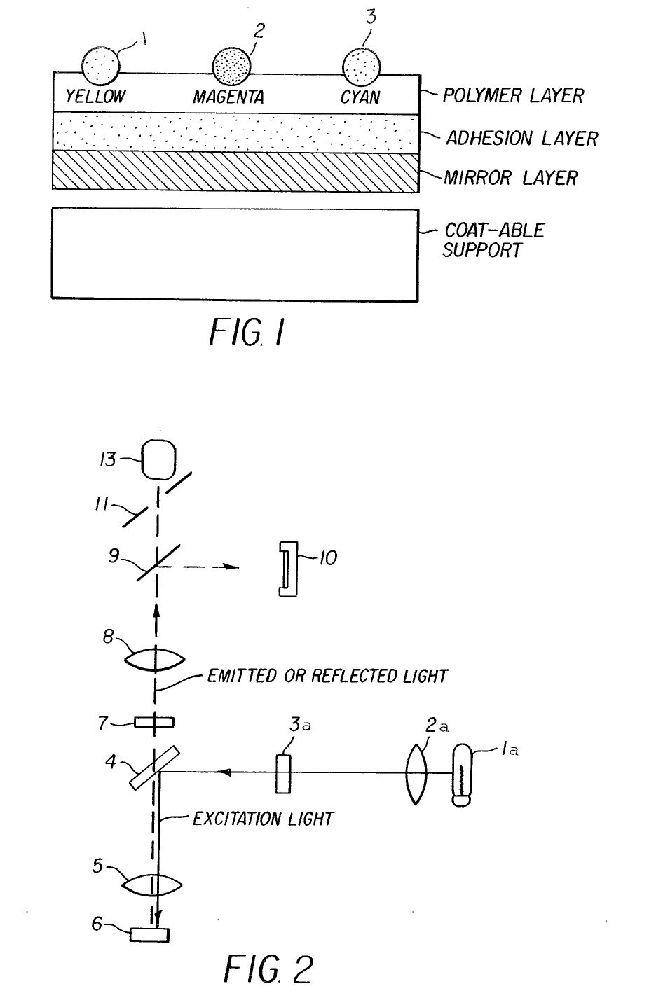

The spectroscopic response of an individual bead is obtained in a microspectrometer set up, consisting of an optical microscope and a spectrophotometer. The procedure for obtaining this response starts by first obtaining a magnified image of the microspheres. This is performed by focusing light from a light source, 1a in FIG. 2, (e.g. halogen or xenon lamp), through the collector lens assembly, 2a, down a dichroic mirror, 4, and onto the microarray specimen, 6. The reflected light is then focused by the objective lens, 5 6, so that a magnified image of a given field of view can be captured. The removable mirror, 9, controls the option of image capture by the digital camera, 10, or spectral capture by the micro-spectrometer, 13. The optical microscope used here is an Olympus BX-30MFSP modular optical system (from Olympus PID Corp, Woodbury, N.Y.), equipped with a Spot RT-Slider Camera (from Diagnostic Instruments, Inc.). Optical microscopy and fluorescence microscopy methods are broadly described by D. B. Murphy, “Fundamentals of Light Microscopy and Electronic Imaging”, Wiley-Liss, Inc. Publishing, 2001; and D. J. Goldstein, “Understanding the Light Microscope. A Computer-aided Introduction”, Academic Press, California, 1999. Depending on the magnification used, optical microscope imaging can provide the location of hundreds to thousands of beads in a single field of view. The combination of many images can provide the location to tens and hundreds of thousands of bead locations.

Once the locations of the microspheres are known, each bead can then be analyzed by spectroscopy by translating the bead of interest, as revealed in the optical microscope image obtained by the procedure described above, into a position for spectral capture. For color analysis of individual microspheres in a random array of mixed color microspheres, our use of UV-VIS spectrometry uniquely allows both the color type and colorant concentration to be obtained. This component is comprised of an F-40 light gathering optics setup (Filmetrics Inc., San Diego) that holds a 45° angled mirror etched with a small aperture, 11 in FIG. 2. This feature permits the extraction of spectral information from a specific region, even from a select region within a 10 micron diameter bead. The spectral information is then collected on the spectrometer sensor, 13 (USB-2000, OceanOptics, Fla.), and processed with the OOIBase32 software (from OceanOptics, Fla.).

Two-dimensional translation of the substrate, containing the microarray, 6 in FIG. 2, allows a bead of interest to be positioned within the spectrometer aperture, 11. Changes in the magnifying power of the objective lens, 5, and the variable zoom lens, 8, allow a different amount of the bead area to be confined by the aperture opening. For analysis of colors in these microspheres, it is preferred that at least two times the area defined by the diameter, D, of the bead is within the aperture opening, i.e. an area of the squared length dimension, π(D/2)², containing the bead of interest. More preferably, one time the area based on the diameter of microsphere is used, and most preferably, 0.5 times the diameter region, in the central portion of the bead, is selected by the aperture opening.

To obtain the color type and color level of colorant in the microsphere, the spectral intensity response in the 300-1000 nm wavelength region of the electromagnetic radiation spectrum is collected, and processed (e.g. as Absorbance, A) following the relationship:

$$Absorbance , A = - log \frac{( {I}_{reference} - {I}_{background} )}{( {I}_{sample} - {I}_{background} )}$$

where
I_{reference}=intensity response of a bead without colorant,
I_{background}=null intensity with zero incident light
Iₛₐₘₚₗₑ=intensity response of colored bead

It is known in the field that the absorbance, A, is related to the concentration of the light absorbing specie in the microsphere by Beer's Law:

A=εbc, where ε is the molar absorptivity of the colorant in the bead,
b is the path length of the microsphere traversed by the light
c is the concentration of the colorant in the bead.

### Therefore,

$$- log \frac{( {I}_{reference} - {I}_{background} )}{( {I}_{sample} - {I}_{background} )} = ɛ b c$$

Therefore, the measured intensity ratio is a monitor of the colorant concentration. The theory and practice of UV-VIS spectroscopy as followed in this disclosure is broadly described by D. A. Skoog and J. J. Leary, in the book “Principles of Analytical Chemistry”, Chapter 6 and 7, Saunders College Publishing, 1992. The processed data can be displayed, for example as Absorbance vs. Wavelength plots, so that changes in colorant spectral response (for identifying its color type), and intensity levels (for identifying color concentration levels) can be collected and evaluated.

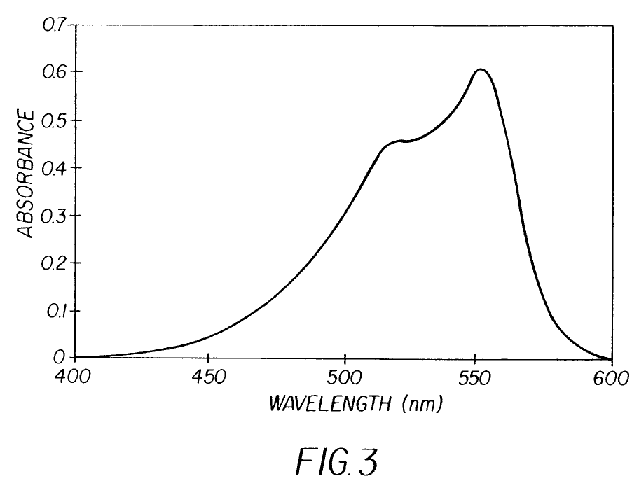

For the invention dye described in this application, its characteristics showed a wavelength response variation with a peak at about 555 nm, and a connected shoulder at about ˜520-530 nm. The full width at half maximum ranged between ˜490-570 nm, FIG. 3. This variation in spectral response over the 400-600 nm range indicates that the colorant in the bead absorbs primarily in the green region of the visible light spectrum.

As shown, the magenta dyes of the present invention are excellent at coloring polystyrene microsphere beads.

### Example 3

This example illustrates the very low fluorescence obtained in polystyrene microsphere beads colored with MD-1

Comparison of the fluorescence intensity variation in several magenta dyes was made using the hybrid analytical system described in Example 2. By incorporating fluorescence cubes, each consisting of an exciter filter, 3a in FIG. 2, a dichroic mirror, 4, and a barrier filter, 7, the fluorescence emission characteristics of a colorant can be monitored. For the magenta dyes used in this invention application, the excitation filter was selected to band pass 545-580 nm, the dichroic mirror had a cut off at 600 nm, and the high pass barrier filter was selected to admit >610 nm light. By setting the spectrometer to collect fluorescence emission intensity for a fixed time, while keeping other experimental conditions constant, comparison of the fluorescence intensity between different dyes were made, Table 1.

TABLE 1
Fluorescence Emission of Colored Beads

| | Maximum Intensity of Fluorescence Emission |
|---|---|
| Dye | (arbitrary units) |
| MD-1 | 215 |
| Comparative Dye 1 | 500 |
| Comparative Dye 2 | greater than 4000 |
| Comparative Dye 3 | 375 |
| Comparative Dye 4 | 450 |

As shown here, the polystyrene micro-spheres have the least fluorescence when imbibed with MD-1.

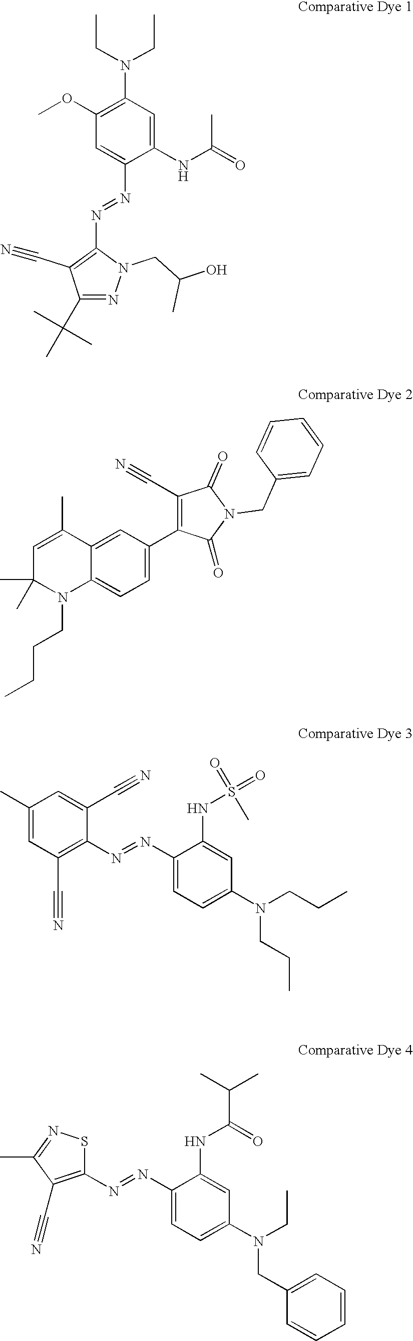

### PARTS LIST

1 yellow colored microsphere
1a light source
2 magenta colored microsphere
2a collector lens assembly
3 cyan colored microsphere
3a exciter filter
4 dichroic mirror
5 objective lens
6 microarray specimen
7 barrier filter
8 variable zoom lens
9 removable mirror
10 digital camera
11 angled mirror
13 microspectrometer

What is claimed is:

1. A microarray comprising:
a substrate coated with a composition comprising
a gelling agent or a precursor to a gelling agent and microspheres; the microspheres containing a dye represented by Formula (I): 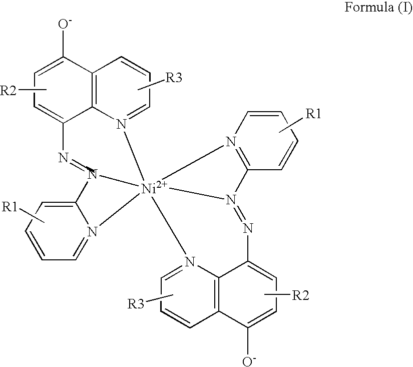
wherein: R1=one or more substitutent selected from the group of H, chloro, alkoxycarbonyl, arylsulfamoyl, or alkylsulfamoyl;
R2=one or more substituent selected from the group of H, carboxamido, or alkoxycarbonyl;
R3=one or more substituent selected from the group of H, chloro, substituted or unsubstituted alkyl, aryl, carboxamido, or alkoxycarbonyl; and
wherein the microspheres are immobilized on the substrate.

2. The microarray according to claim 1 wherein the microspheres contain a dye represented by Formula (II): 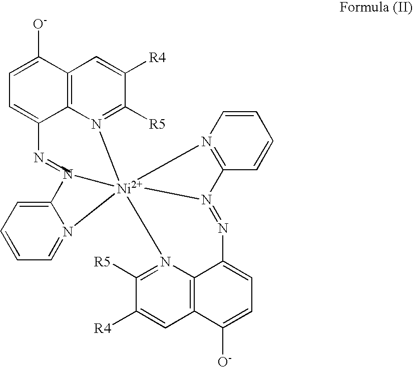
wherein: R4=alkoxycarbonyl; and
R5=H, chloro, or alkyl.

3. The microarray according to claim 1 wherein the gelling agent is gelatin.

4. The microarray according to claim 1 wherein the microspheres bear chemically active sites.

5. The microarray according to claim 4 wherein the chemically active site is bioactive.

6. A microarray according to claim 1 wherein the substrate comprises glass, plastic, cellulose acetate, or polyethyleneterephthalate.

7. The microarray according to claim 1 wherein the substrate is flexible.

8. The microarray according to claim 1 wherein the microspheres are immobilized on the substrate in a concentration between 100 and 1 million microspheres per cm².

9. The microarray according to claim 1 wherein the microspheres are immobilized on the substrate in a concentration between 1000 and 200,000 microspheres per cm².

10. The microarray according to claim 1 wherein the microspheres are immobilized on the substrate in a concentration between 10,000 and 100,000 microspheres per cm².

11. The microarray according to claim 1 wherein the microspheres are immobilized on the substrate upon gelation of the gelling agent.

12. The microarray according to claim 1 wherein the microspheres carry surface active sites.

13. The microarray according to claim 12 wherein the surface active sites carry organic or inorganic attachments.

14. A coating composition according to claim 12 wherein the surface active site is capable of chemical or physical interaction.

15. The microarray according to claim 12 wherein the surface active site is bioactive.

16. The microarray according to claim 15 wherein the bioactive site interacts with nucleic acid, protein, or fragments thereof.

17. The microarray of claim 1 wherein the microspheres are randomly distributed on the substrate.

18. The microarray of claim 1 wherein the substrate is characterized by an absence of specific sites capable of interacting physically or chemically with the microspheres.

19. A microsphere for making an array, the microsphere comprising a capsule containing a dye represented by Formula (I).

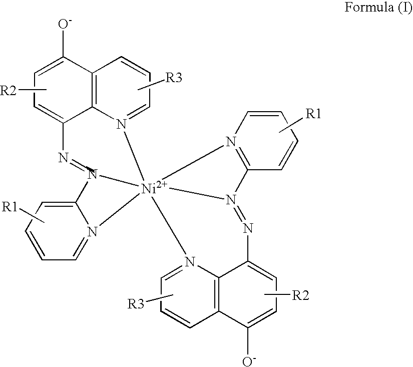

20. A microsphere for making an array, the microsphere comprising a capsule containing a dye represented by Formula (II): [chemical structure]
